(19) United States
(12) Patent Application Publication
(10) Pub. No.: US 2014/0296743 A1
(43) Pub. Date: Oct. 2, 2014

(54) PORTABLE LASER BLOOD SAMPLING DEVICE HAVING DUAL SAFETY DEVICE AND DISPOSABLE CAP USED THEREIN

(71) Applicant: Kee Jung CHOI, Daejeon (KR)

(72) Inventor: Kee Jung CHOI, Daejeon (KR)

(21) Appl. No.: 13/854,170

(22) Filed: Apr. 1, 2013

(51) Int. Cl. A61B 5/151

(52) U.S. Cl. CPC A61B 5/15138
USPC 600/573

(57) ABSTRACT

A portable blood sampling device comprises: a laser module; a skin contact part which is disposed in the direction in which the laser radiation is emitted so that the laser radiation is locally concentrated on a region of a skin that is to be perforated; a radiation port cover disposed through the laser module and across the direction in which the laser radiation is emitted; a first safety switch part which switches on and off via upward and downward movement in response to opening and closing of the radiation port cover; a second safety switch part which has an elastic member between the laser module and the skin contact part and switches on and off in response to pushing and pulling of the skin contact part; and a button switch which allows the laser radiation to be emitted.

### BACKGROUND OF THE INVENTION

1. Field of the Invention

The present invention relates, in general, to a portable laser blood sampling device having a dual safety device, which obtains a small amount of blood sample by stimulating a capillary vessel under the skin using an Er-YAG laser, and a disposable cap used therein.

2. Description of the Related Art

In general, an erbium-doped yttrium aluminum garnet (Er-YAG) laser used for painless blood sampling has a reflector part which is made by depositing or plating the surface of a metal piece with Ag, Ni, Cu or an alloy thereof several times and machining the resultant structure. A flash lamp and an Er-YAG crystal rod which generate laser radiation are disposed inside the reflector part.

The intensity of laser radiation emitted from the Er-YAG laser varies depending on the capacity of a photocapacitor which determines an input voltage of the flash lamp, the amount of radiation of the flash lamp, and the volume, length and density of the Er-YAG crystal rod.

The laser blood sampling device having a laser module as above may cause a fatal problem to a user in a general situation or when the laser module malfunctions.

Therefore, the size of the blood sampling device must be reduced such that it can be used by a diabetic whose blood must be sampled several times a day so that the laser blood sampling device can be provided for the convenience of a user. However, even if the size of the laser module is reduced, several safety devices must be provided on the exterior of the blood sampling device in order to prevent the laser module from malfunctioning. In such a case, the laser blood sampling device is not substantially miniaturized.

In addition, when a focus lens which collects laser radiation that is emitted through the laser module is provided, impurities such as smoke may be produced when a target region of the user is irradiated with the laser radiation from the laser module.

### SUMMARY OF THE INVENTION

Accordingly, the present invention has been made keeping in mind the above problems occurring in the related art, and the present invention is intended to propose a laser blood sampling device, the size of which is reduced such that it can be carried around, and which is provided with a double safety device in order to prevent a laser module from malfunctioning.

The present invention is also intended to propose an optical window or a disposable cap to protect a focus lens which collects laser radiation emitted from the laser module.

In order to achieve the above object, according to one aspect of the present invention, there is provided a portable blood sampling device which includes: a laser module which generates laser radiation and emits the laser radiation in one direction; a skin contact part which is disposed in the direction in which the laser radiation is emitted so that the laser radiation is locally concentrated on a region of a skin that is to be perforated; a radiation port cover disposed through the laser module and across the direction in which the laser radiation is emitted; a first safety switch part which switches on and off via upward and downward movement in response to opening and closing of the radiation port cover; a second safety switch part which has an elastic member between the laser module and the skin contact part and switches on and off in response to pushing and pulling of the skin contact part; and a button switch which allows the laser radiation to be emitted when the first safety switch part and the second safety switch part are switched on.

According to an embodiment of the portable blood sampling device, the skin contact part may include: a disposable cap insertion port which is formed coaxially with the radiation port cover, such that a disposable cap is to be inserted into the disposable cap insertion port; and an optical window insertion port into which an optical window is to be inserted, the optical window protecting a focus lens of the laser module from impurities caused by the laser radiation emitted from the laser module.

The optical window may be fixed to or be attachable to and detachable from the optical window insertion port.

The portable blood sampling device may further include: a laser intensity adjusting button with which the intensity of the laser radiation emitted from the laser module is adjusted; and a battery level indicator which indicates a battery level at which the laser module is operable.

The skin contact part may further include a push part which elastically switches on and off the second safety switch part.

According to another aspect of the present invention, there is provided a disposable cap used in a laser blood sampling device which includes: a coupling portion which is configured so as to be coupled to a disposable cap insertion port of a skin contact part of the laser blood sampling device; a through-hole portion which is formed in the coupling portion, and has a hollow inner portion and open both ends such that laser radiation is emitted outward via the through-hole portion; and a knob portion with which the coupling portion is coupled to the skin contact part, the coupling portion, the through-hole portion and the knob portion being integrally formed.

According to an embodiment of the disposable cap, one side of the through-hole portion may have defined therein a transparent interface film which prevents the laser blood sampling device from being contaminated by blood of a user and a lens portion which is positioned between the transparent interface film and a user contact surface, the lens portion focusing the laser radiation onto an outside region from which a blood sample is to be taken.

The knob portion may further have an anti-slip portion which prevents the knob from slipping when the user separates or couples the coupling portion from or to the skin contact part.

The though-hole portion may have a shape of a hollow cylinder having both ends open, and be perpendicular to the coupling portion.

According to embodiments of the present invention as set forth above, since the radiation port cover or the double safety switch parts are provided inside the case section, it is possible to prevent a malfunction in which laser radiation is emitted to the outside when the blood sampling device is not in contact with a target region of the skin.

Also, since the safety parts which were previously provided on the exterior of the laser blood sampling device are excluded and instead disposed inside the case section, the effect of the reduced size of the laser blood sampling device is achieved.

Furthermore, since the disposable cap has the lens depending on whether or not the laser module has the focus lens, there is the effect of the increased efficiency of the laser radiation.

### BRIEF DESCRIPTION OF THE DRAWINGS

The above and other objects, features and advantages of the present invention will be more clearly understood from the following detailed description when taken in conjunction with the accompanying drawings, in which:

FIG. 1 is a perspective view showing a portable laser blood sampling device having a double safety device according to an embodiment of the present invention;

FIG. 2 is a perspective view showing a first safety switch part and a second safety switch part of the portable laser blood sampling device having the double safety device according to an embodiment of the present invention;

FIG. 3a and FIG. 3b are perspective views showing the portable laser blood sampling device having the double safety device according to an embodiment of the present invention and a portable cap coupled thereto;

FIG. 4a and FIG. 4b are front elevation views showing a cover of a radiation port of the portable laser blood sampling device having the double safety device according to an embodiment of the present invention;

FIG. 5 is a bottom perspective view showing a skin contact part of the portable laser blood sampling device having the double safety device according to an embodiment of the present invention;

FIG. 6 is a perspective view showing a disposable cap which is used in a portable laser blood sampling device according to an embodiment of the present invention; and

FIG. 7 is a cross-sectional view showing the disposable cap which is used in the portable laser blood sampling device according to an embodiment of the present invention.

### DETAILED DESCRIPTION OF THE INVENTION

Reference will now be made in greater detail to a preferred embodiment of the present invention, an example of which is illustrated in the accompanying drawings. Wherever possible, the same reference numerals will be used throughout the drawings and the description to refer to the same or like parts.

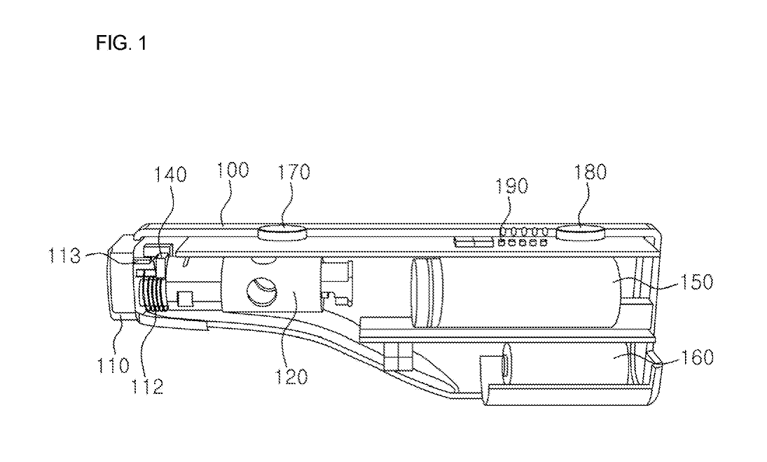

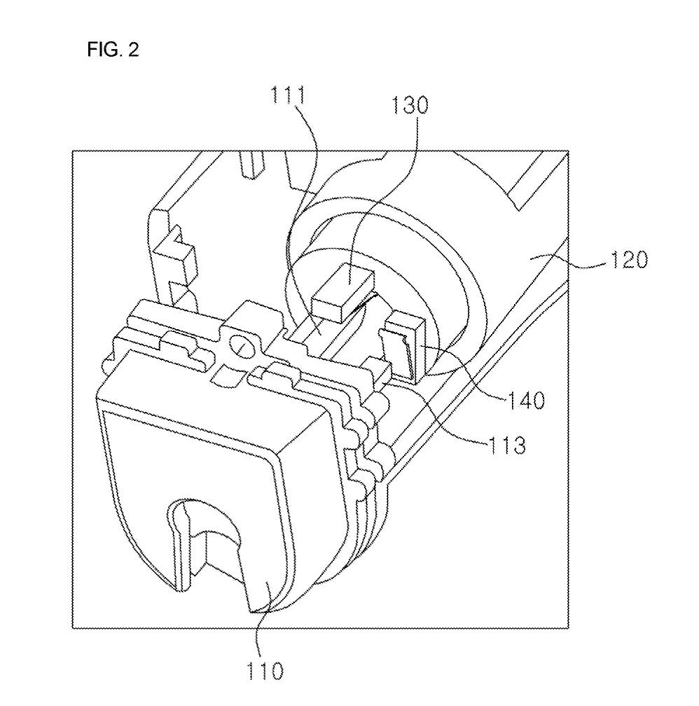

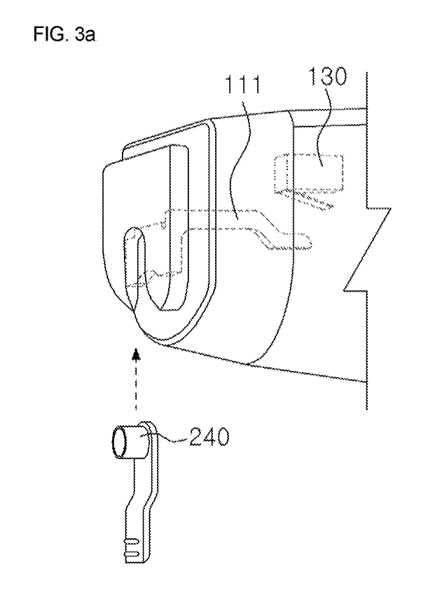

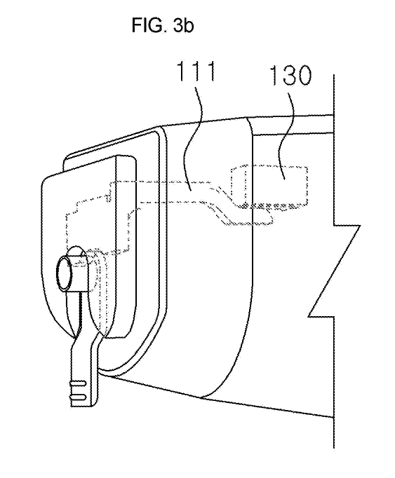

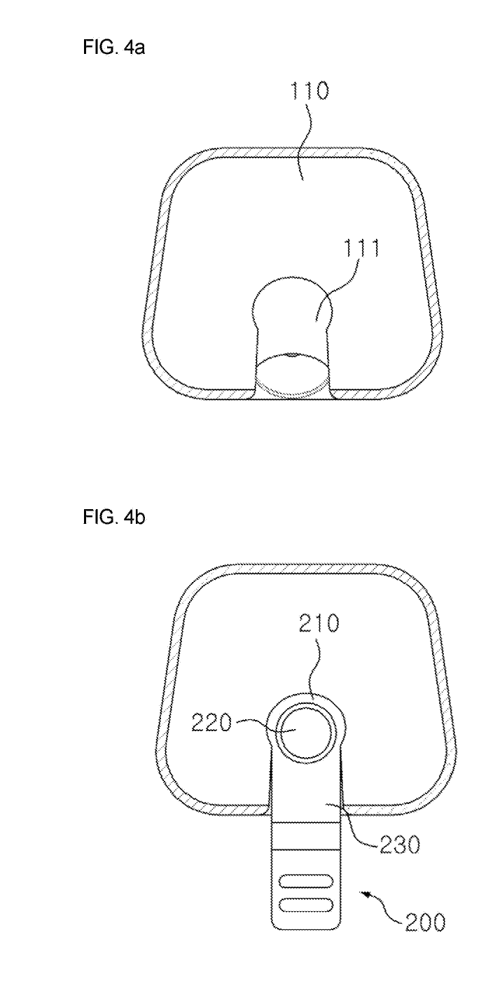

FIG. 1 is a perspective view showing a portable laser blood sampling device having a double safety device according to an embodiment of the present invention, FIG. 2 is a perspective view showing a first safety switch part and a second safety switch part of the portable laser blood sampling device having the double safety device according to an embodiment of the present invention, FIGS. 3a and 3b are perspective views showing the portable laser blood sampling device having the double safety device according to an embodiment of the present invention and a portable cap coupled thereto, FIGS. 4a and 4b are front elevation views showing a cover of a radiation port of the portable laser blood sampling device having the double safety device according to an embodiment of the present invention, and FIG. 5 is a bottom perspective view showing a skin contact part of the portable laser blood sampling device having the double safety device according to an embodiment of the present invention.

As shown in FIG. 1 to FIG. 5, the portable laser blood sampling device having the double safety device according to an embodiment of the present invention includes a skin contact part 110, a laser module 120, a first safety switch part 130, a second safety switch part 140, a button switch part 170, a laser intensity adjusting part 180, and a battery level indicator 190.

The laser module 120, the first safety switch part 130 and the second safety switch part 140 are positioned inside a case section 100, and the button switch part 170, the laser intensity adjusting part 180 and the battery level indicator 190 are positioned at outer portions of the case section 100 which a user can easily touch.

In addition, the skin contact part 110 is positioned on the front portion of the case section in the direction in which laser radiation from the laser module 120 is emitted, and has elasticity due to an elastic member 112 between the case section 100 and the skin contact part 110.

Thus, even if a user has pushed the skin contact part 110 in the direction toward the case section 100, the elastic member 112 returns the skin contact part 110 to the original position when the user no longer touches the skin contact part 110.

A description will be given below of the configuration of the portable laser blood sampling device having the double safety device according to an embodiment of the present invention.

The laser module 120 is a device which generates laser radiation and emits it in one direction. However, the laser module 120 will be described in brief, since it is not a key part of the present invention.

Here, the laser module 120 includes a reflector, an Er-YAG crystal rod and a flash lamp, which are disposed inside the reflector and are exposed to the outside, and a metal guide and a silicone guide, which fix the Er-YAG crystal rod and the flash lamp in an inner space of the reflector and absorb external impacts.

Both ends of the Er-YAG crystal rod and the flash lamp support components, which generate the Er-YAG laser radiation, in a front part fixing cover and a rear part fixing cover, which are respectively disposed at both sides of the front end and the rear end of the reflector. Provided are a coupling which connects and fixes the front part fixing cover and the rear part fixing cover, a focusing lens which focuses the laser radiation at a correct focus on the skin at a region from which blood is to be sampled, and a lens bushing which fixes the focusing lens.

In addition, a semi-transmission mirror is attached to the front surface of the Er-YAG crystal rod, and a total-reflection mirror is attached to the rear surface of the Er-YAG crystal rod.

The Er-YAG crystal rod is a component which guides and condenses a flashlight generated from the flash lamp, thereby generating laser radiation that has an intensity ranging from 50 mJ to 350 mJ and a wavelength of about 2.94 μm. The intensity of the laser radiation is determined depending on the crystalline structure, density and volume of the Er-YAG crystal rod, the amount of radiation of the flash lamp, and the charge voltage and capacity of a photocapacitor 150 which determines the output power of the flash lamp.

The reflector in which the Er-YAG crystal rod and the flash lamp are disposed at a predetermined distance from each other is a component which forms the outer shape of an Er-YAG laser generating unit. The inner surface of the reflector reflects at least 90% of a flashlight generated from the flash lamp, and guides and condenses the flashlight to the Er-YAG crystal rod.

The laser module 120 having the above-described configuration generates laser radiation and emits it in one direction, thereby helping the user to obtain a blood sample from a finger. The laser radiation generated by the laser module 120 is laser radiation having a wavelength of 2.94 μm, which allows the blood sample to be taken from the skin painlessly based on the Er-YAG laser technology, and causes substantially no harm to the human body due to its absorptivity to water.

Although the use of the Er-YAG laser technology has been described as an embodiment of the present invention, the present invention is not limited thereto. Any technology which is harmless to the human body and has characteristics similar to those of the Er-YAG laser technology can of course be used.

The skin contact part 110 is disposed in the direction in which laser radiation is emitted such that the laser radiation can be locally concentrated on the region of the skin that is to be perforated.

Here, the skin contact part 110 includes a disposable cap insertion port 115, a radiation port cover 111, a push part 113 and an optical window insertion port 114.

The disposable cap insertion port 115 is formed coaxially with the radiation port cover 111 such that a disposable cap 200 can be inserted thereinto. The disposable cap insertion port 115 has a guide which allows the disposable cap 200 to be inserted so that a through-hole 220 of the disposable cap 200 is positioned collinear with the direction in which the laser module 120 emits the laser radiation.

The radiation port cover 111 is disposed through the disposable cap insertion port 115 and across the direction in which the laser module 120 emits the laser radiation. The radiation port cover 111 moves up and down by the disposable cap 200 which is inserted through the disposable cap insertion port 115. The radiation port cover 111 extends further into the skin contact part 110.

The push part 113 extends coaxially with the case section 100 inside the skin contact part 110, and controls the second safety switch part 140.

The optical window insertion port 114 allows an optical window 300 to be inserted thereinto. The optical window 300 serves to block impurities such as fine drops of blood, which occur during blood sampling using the laser radiation emitted from the laser module 120, from splashing onto a focusing lens (not shown) of the laser module 300 positioned inside the case section 100.

Here, the optical window 300 can have a structure that is formed integrally with the portable laser blood sampling device, or a structure that is attachable to and detachable from the portable laser blood sampling device. When the optical window 300 has the latter structure, an additional configuration which allows the optical window 300 to be easily attached to and detached from the blood sampling device can also be employed.

In addition, materials for the optical window 300 are not limited to glass, but also include plastic and any transparent materials which do not cause noise when the laser radiation is sent outward.

The first safety switch part 130 is a first release device which allows the laser module 120 to operate. The first safety switch part 130 switches on and off in response to upward and downward movement of the radiation port cover 111 which further extends into the skin contact part 110.

In other words, the switch of the first safety switch part 130 is positioned on the top of the radiation port cover 111, such that the radiation port cover 111 touches the switch of the first safety switch part 130 while moving up.

According to this embodiment of the present invention, when the radiation port cover 111, which moves in order to open in the direction in which the laser radiation is emitted, moves up, the first safety switch part 130 is positioned on the top of the radiation port cover 111 which extends into the skin contact part 110.

However, when the radiation port cover 111, which moves in order to open in the direction in which the laser radiation is emitted, moves down, it is preferred that the first safety switch part 130 be positioned on the bottom of the radiation port cover 111 which extends into the skin contact part 110.

The second safety switch part 140 is a second release device which allows the laser module 120 to operate. The second safety switch part 140 switches on and off in response to pushing and pulling of the push part 113 which extends coaxially with the case section 100 inside the skin contact part 110.

In other words, the second safety switch part 140 is released by pushing the skin contact part 110 which is elastically connected to the laser module 120 via the elastic member 112.

In addition, the state in which the skin contact part 110 is not pushed and has no contact with the user is referred to as the pulled state.

The button switch part 170 allows the laser module to operate so that the laser radiation is emitted to the outside. The button switch part 170 can operate in the state in which both the first safety switch part 130 and the second safety switch part 140 are released.

In other words, neither the first safety switch part 130 nor the second safety switch part 140 is released (switched on), it is impossible to operate the laser module 120 with the button switch 170.

Therefore, it is possible to reduce the risk of dangerous problems caused by malfunctioning of the laser module 120.

The laser intensity adjusting part 180 adjusts the intensity of emission of the laser module 120. Since the thickness of the skin varies depending on the user, the intensity of the laser radiation is adjusted to an intensity level that is suitable to each user.

The battery level indicator 190 indicates a battery level at which the laser module can operate.

According to this embodiment of the present invention relating to the portable laser blood sampling device, the battery level can be indicated such that the blood sampling device can be used in any place.

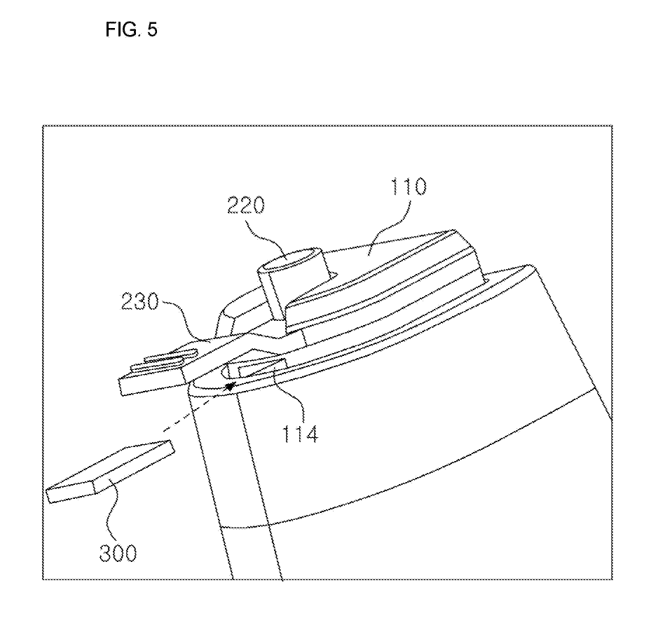

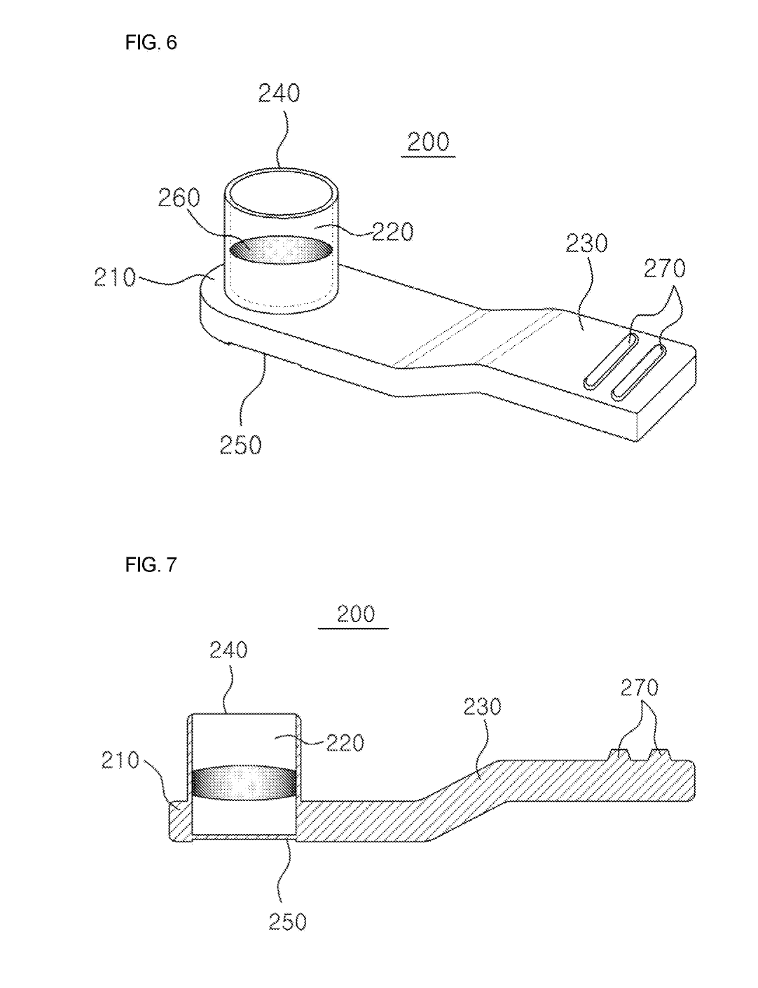

FIG. 6 is a perspective view showing the disposable cap which is used in the portable laser blood sampling device according to an embodiment of the present invention, and FIG. 7 is a cross-sectional view showing the disposable cap which is used in the portable laser blood sampling device according to an embodiment of the present invention.

As shown in FIG. 6 and FIG. 7, the disposable cap 200 which is used in the portable laser blood sampling device according to the present invention includes a coupling portion 210, a through-hole portion 220, a knob portion 230 and a lens portion 260.

The coupling portion 210 is a portion by which the disposable cap 200 is fixed to the skin contact part 110 of the portable laser blood sampling device. The coupling portion 210 can have any structure that does not obstruct laser radiation emitted from the laser blood sampling device.

The through-hole portion 220 includes a contact surface 240 which comes into contact with the skin to which the laser radiation is to be emitted when the laser radiation generated by the laser module 120 is emitted outward and a transparent interface film 250 which prevents the laser blood sampling device from being contaminated by the blood of the user. The through-hole portion 220 has the shape of a hollow cylinder having open ends, and is formed perpendicular to the coupling portion 210.

The transparent interface film 250 prevents the dispersion of the blood or smoke which would occur when the blood sample is being taken, prevents the focusing lens from being contaminated, and overcomes the problem of infection being transmitted between users as a result of reuse.

Although the through-hole portion 220 has been illustrated as having the shape of a hollow cylinder having open ends in this embodiment of the present invention, this shape is a structure that can allow the laser radiation to be efficiently emitted outward. However, this structure is not intended to be limiting.

The knob portion 230 is formed integrally with the coupling portion 210 such that the user can couple the coupling portion 210 to the skin contact part 110 by holding the knob portion 230.

Here, it is preferred that the knob portion 230 also has an anti-slip portion 270 which prevents the knob portion 230 from slipping so that the user does not fumble the disposable cap 200 when the user couples or separates the coupling portion 210 to or from the disposable cap insertion port 115 of the skin contact part 110.

Although the portion between the knob portion 230 and the coupling portion 210 is inclined at an angle in this embodiment of the present invention, this is an ergonomic structure that facilitates coupling of the coupling portion 210 to the disposable cap insertion port 115 of the skin contact part 110. Rather, the present invention is not limited to the angled structure.

The lens portion 260 is positioned between the transparent interface film 250 and the contact surface 240, and focuses the laser radiation onto the region of the body from which the blood is to be sampled.

The disposable cap 200 may not include the lens portion 260 when a focus lens coupled to the portable laser blood sampling device is provided.

This is because, when both the focus lens and the lens portion 260 of the disposable cap 200 are provided, the efficiency of laser radiation that has passed through the two lenses decreases.

A description will be given below of the process of mounting the disposable cap 100 having the above-described configuration and the route of the laser radiation.

The user holds the knob portion 230 and couples the coupling portion 210 to the disposable cap insertion port 115 of the skin contact part 110.

When the disposable cap 200 is coupled to the disposable cap insertion port 115 as described above, the laser module 120 generates laser radiation and emits it outward. The laser radiation passes through the transparent interface film 250 and is focused by the lens portion 240 before irradiating the skin of the user.

Consequently, the disposable cap 200 is in contact with the target region of the user, thereby reducing the distance between the target region and the laser module. As advantageous effects, it is possible to reduce smell, smoke and noise that occur when perforating the skin.

A description will be given below of the sequence of the operation of the portable laser blood sampling device having the double safety device according to an embodiment of the present invention.

First, the disposable cap 200 is fitted into the disposable cap insertion port 115 while pushing the radiation port cover 111 up using the disposable cap 200. Here, the through-hole portion 220 of the disposable cap 200 is disposed coaxially with the direction of laser radiation emitted from the laser module 120.

Afterwards, the disposable cap 200 pushes the radiation port cover 111 so that the radiation port cover 111 which extends into the skin contact part 110 releases the first safety switch part 130 inside the case section 100 while moving up.

In sequence, the battery level is checked using the battery level indicator 190 in order to determine whether or not the laser module 120 can operate, and the intensity of emission of the laser module is adjusted using the laser intensity adjusting button 180.

After that, a target region of the skin of the user, i.e. a finger from which a blood sample is to be taken, is brought into contact with the contact surface 240 of the disposable cap 200, and is pressed against the skin contact part 140.

At this time, the push part 113 which extends coaxially with the case section 100 inside the skin contact part 110 presses and releases the second safety switch part 140.

Finally, when both the first and second safety switch parts 130 and 140 are released, the laser module 120 is operated using the button switch 170.

While the present invention has been described in connection with the exemplary embodiments of the present invention, it is apparent to a person having ordinary skill in the art that various modifications and changes can be made without departing from the scope of the present invention. Therefore, it is to be understood that the scope of the present invention is not limited to the foregoing embodiments but shall be defined by the accompanying claims and their equivalents.

What is claimed is:

1. A portable blood sampling device, comprising:
a laser module which generates laser radiation and emits the laser radiation in one direction;
a skin contact part which is disposed in the direction in which the laser radiation is emitted so that the laser radiation is locally concentrated on a region of a skin that is to be perforated;
a radiation port cover disposed through the laser module and across the direction in which the laser radiation is emitted;
a first safety switch part which switches on and off via upward and downward movement in response to opening and closing of the radiation port cover;
a second safety switch part which has an elastic member between the laser module and the skin contact part and switches on and off in response to pushing and pulling of the skin contact part; and
a button switch which allows the laser radiation to be emitted when the first safety switch part and the second safety switch part are switched on.

2. The portable blood sampling device according to claim 1, wherein the skin contact part comprises:
a disposable cap insertion port which is formed coaxially with the radiation port cover, such that a disposable cap is to be inserted into the disposable cap insertion port; and
an optical window insertion port into which an optical window is to be inserted, the optical window protecting a focus lens of the laser module from impurities caused by the laser radiation emitted from the laser module.

3. The portable blood sampling device according to claim 2, wherein the optical window is fixed to or is attachable to and detachable from the optical window insertion port.

4. The portable blood sampling device according to claim 1, further comprising:
a laser intensity adjusting button with which an intensity of the laser radiation emitted from the laser module is adjusted; and
a battery level indicator which indicates a battery level at which the laser module is operable.

5. The portable blood sampling device according to claim 1, wherein the skin contact part further comprises a push part which elastically switches on and off the second safety switch part.

6. A disposable cap used in a laser blood sampling device, comprising:
a coupling portion which is configured so as to be coupled to a disposable cap insertion port of a skin contact part of the laser blood sampling device;
a through-hole portion which is formed in the coupling portion, and has a hollow inner portion and open both ends such that laser radiation is emitted outward via the through-hole portion; and
a knob portion with which the coupling portion is coupled to the skin contact part, the coupling portion, the through-hole portion and the knob portion being integrally formed, wherein
one side of the through-hole portion has defined a transparent interface film which prevents the laser blood sampling device from being contaminated by blood of a user and a lens portion which is positioned between the transparent interface film and a user contact surface, the lens portion focusing the laser radiation onto an outside region from which a blood sample is to be taken.

7. The disposable cap according to claim 6, wherein the knob portion further has an anti-slip portion which prevents the knob from slipping when the user separates or couples the coupling portion from or to the skin contact part.

8. The disposable cap according to claim 6, wherein the though-hole portion has a shape of a hollow cylinder having both ends open, and is perpendicular to the coupling portion.
